(19) United States
(12) Patent Application Publication
(10) Pub. No.: US 2026/0118460 A1
(43) Pub. Date: Apr. 30, 2026

(54) NOISE DAMPENING FOR INTENSITY CORRECTION IN MAGNETIC RESONANCE IMAGING

(71) Applicant: CANON MEDICAL SYSTEMS CORPORATION, Otawara-shi (JP)

(72) Inventor: Andrew J WHEATON, Vernon Hills, IL (US)

(73) Assignee: CANON MEDICAL SYSTEMS CORPORATION, Otawara-shi (JP)

(21) Appl. No.: 18/926,618

(22) Filed: Oct. 25, 2024

(51) Int. Cl. G01R 33/56; G01R 33/24; G01R 33/565; G16H 30/40

(52) U.S. Cl. CPC G01R 33/5608; G01R 33/246; G01R 33/5659; G16H 30/40

(57) ABSTRACT

A method, system and computer program product for providing noise suppression in imaging data (e.g., magnetic resonant imaging (MRI) data) based on a correction factor related to an image intensity uniformity function (e.g., a sensitivity map) and an intensity correction function that varies with the signal intensities of the imaging data.

### BACKGROUND OF THE INVENTION

### Field of the Invention

A method, system, processing circuitry, and computer program product for providing image processing in medical images, and in one embodiment, to a method, system and computer program product for providing a reduction in the appearance of noise in low intensity image areas in magnetic resonance imaging images.

### Discussion of the Background

In Magnetic Resonance Imaging (MRI) systems, MR receiver (Rx) elements often include multiple Rx coils. The local reception field of each Rx element is non-uniform according to the Biot-Savart Law given by:

$$B ( r ) = \frac{{μ}_{0}}{4 π} {\int }_{C} \frac{Idl \times {r}^{′}}{{\vert {r}^{′} \vert}^{3}} \qquad ( Eq . 1 )$$

Using data from uncorrected, non-uniform Rx fields produces a final image with non-uniform signal intensity which often is undesirable for physicians. As shown in FIG. 1A, typically, the intensity is greater in portions of the scanned object (e.g., within the white box) that are closer to the receiver element than portions (e.g., within the circle) that are farther from the receiver element. To address this non-uniformity, a map of correction factors, sometimes called a sensitivity map, can be created and applied to an original, uncorrected image. In one embodiment, the sensitivity map is created using a two-part prescan process. In one part, a first image is generated from a whole-body coil (WBC) which is assumed to have a uniform receiver intensity. In the other part of the prescan, a phased array coil (PAC) is used to generate a second image. The sensitivity map (S(x)) is then generated as follows:

$$S ( x ) = \frac{P A C ( x )}{W B C ( x )} . \qquad ( Eq . 2 )$$

Alternatively, the PAC map can be normalized by the sum-of-squares of all channels or by unity (1.0 value everywhere). Intensity correction can then be performed on the original image by dividing the original image by the sensitivity map as shown below.

$${I}_{c orrected} ( x ) = \frac{{I}_{o r i g i n a l} ( x )}{S ( x )} \qquad ( Eq . 3 )$$

As a result, a more uniform image is created, as shown in FIG. 1B. However, by applying the above correction, the noise in regions with low sensitivity are amplified as shown within the white oval of FIG. 1B.

As can be seen by comparing the original image of FIG. 1C to a corrected image of FIG. 1D, the noise amplification problem is exacerbated when there is a dark signal in the interior of the image. This situation can be caused by tissue suppression by inversion recovery (e.g. FLAIR, STIR, or SPAIR), saturation (e.g. CHESS, spatial presaturation), background tissue signal suppression (e.g. Time of Flight angiography), or subtraction (e.g. Arterial Spin Labeling). The noise-amplified image appears to be noisy and visually unpleasing, and the noise amplification is also noticeable in the air surrounding the body.

This same problem can happen for FLAIR images of the brain, as shown by a comparison of an original image of FIG. 1E to a corrected image of FIG. 1F. As seen in FIG. 1F, the cerebrospinal fluid (CSF) in a corrected image can get ‘milky’ due to noise amplification. Also, the air surrounding the head can be amplified, causing unpleasing image quality as well.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete appreciation of the disclosure and many of the attendant advantages thereof will be readily obtained as the same becomes better understood by reference to the following detailed description when considered in connection with the accompanying drawings, wherein:

FIG. 1A is a first exemplary original image showing image non-uniformity caused by proximity to a receiver element where the intensity is greater in portions of the scanned object (e.g., within the white box) that are closer to the receiver element than portions (e.g., within the circle) that are farther from the receiver element;

FIG. 1B is a first corrected image generated by applying a sensitivity map to the original image of FIG. 1A;

FIG. 1C is a second exemplary image in which there is a dark signal in the interior of the image;

FIG. 1D is a second corrected image resulting from a correction of the image of FIG. 1C but having visually unpleasing amplified noise in the area corresponding to the dark signal in the interior of the image;

FIG. 1E is a third exemplary image obtained using Fluid-attenuated inversion recovery (FLAIR);

FIG. 1F is a third corrected image resulting from a correction of the image of FIG. 1E but having visually unpleasing amplified noise in an area corresponding to the cerebrospinal fluid (CSF);

FIG. 2 is a schematic of an MRI apparatus;

FIG. 3 is a flowchart showing a generalized process of correcting an image as described herein;

FIGS. 4A to 4D are graphs of exemplary intensity correction functions;

FIG. 5 is a graph of an exemplary intensity function for use with complex or signed magnitude data;

FIG. 6 is a graph of an exemplary intensity correction function having a dynamically calculated critical transition point;

FIGS. 7A and 7B are images generated (a) using only a sensitivity map and (b) using a sensitivity map and an intensity correction function, respectively;

FIG. 7C is a color difference image showing differences in signal intensities between FIGS. 7A and 7B;

FIGS. 8A and 8B are additional images generated (a) using only a sensitivity map and (b) using a sensitivity map and an intensity correction function, respectively;

FIG. 8C is a color difference image showing differences in signal intensities between FIGS. 8A and 8B; and

FIGS. 9A and 9B are illustrations of graphical user interfaces for selecting an intensity correction function to be applied to an image that is being corrected.

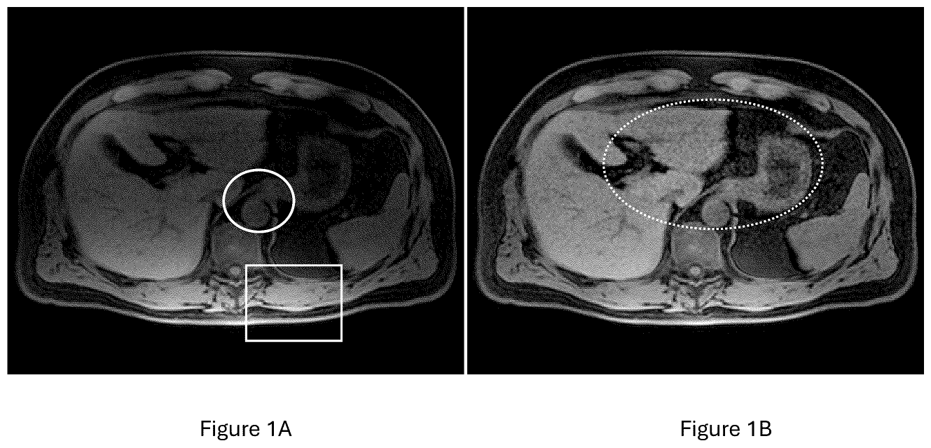

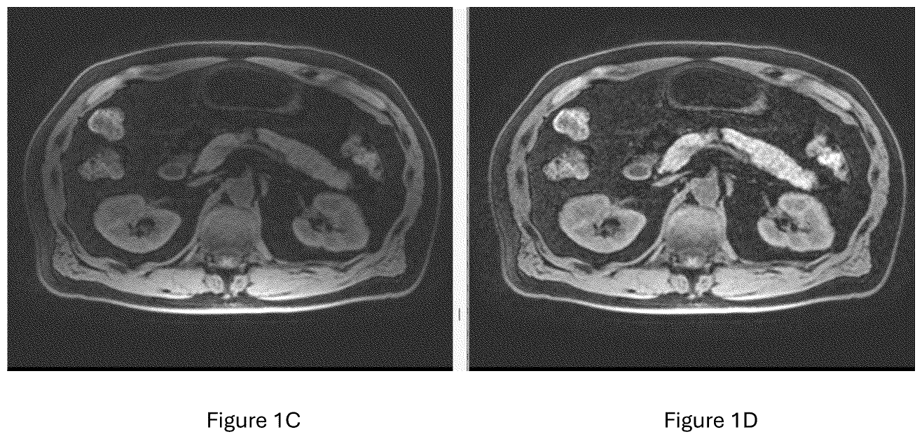

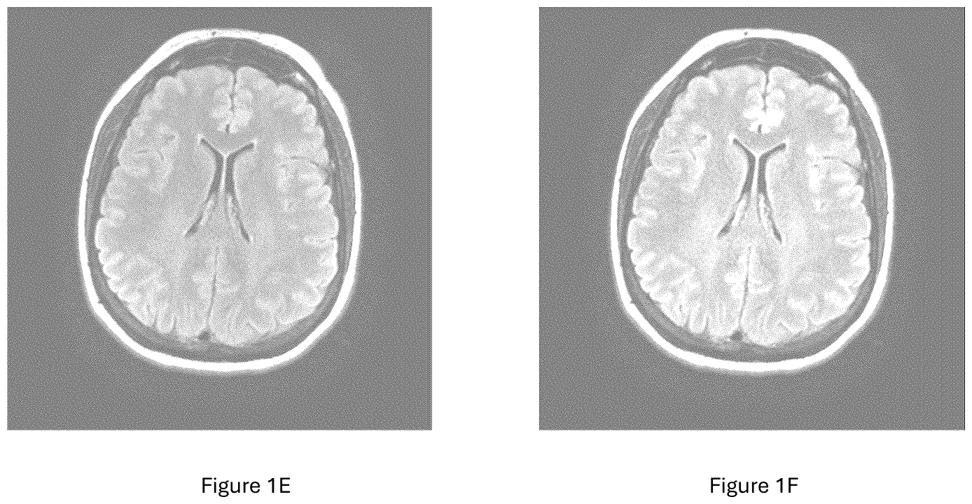

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The present disclosure is related to a method, system, and non-transitory computer-readable storage medium storing computer-readable instructions for reducing the appearance of noise in imaging data (e.g., magnetic resonant imaging (MRI) data) based on (a) a correction factor related to a sensitivity map and (b) an intensity correction function that varies with the signal intensities of the imaging data.

In one embodiment, it can be appreciated that the present disclosure can be viewed as a system. While the present exemplary embodiments will refer to an MRI apparatus, it can be appreciated that other system configurations can use other medical imaging apparatuses (e.g., CT systems and combined MRI/CT systems).

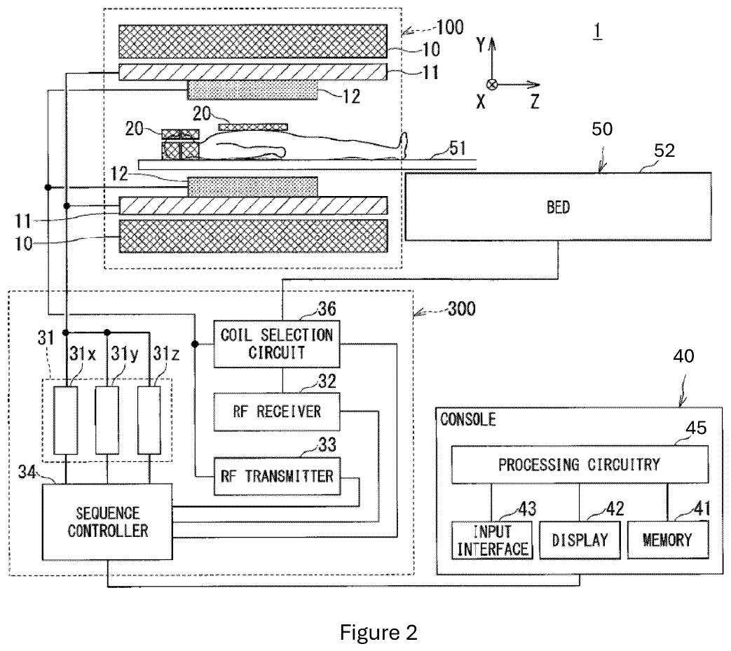

Referring now to the drawings, FIG. 2 is a block diagram illustrating overall configuration of an MRI apparatus 1. The MRI apparatus 1 includes a gantry 100, a control cabinet 300, a console 40, a bed 50, and radio frequency (RF) coils 20. The gantry 100, the control cabinet 300, and the bed 50 constitute a scanner, i.e., an imaging unit.

The gantry 100 includes a static magnetic field magnet 10, a gradient coil 11, and a whole-body (WB) coil 12, and these components are housed in a cylindrical housing. The bed 50 includes a bed body 52 and a table 51.

The control cabinet 300 includes three gradient coil power supplies 31 (31 x for an X-axis, 31 y for a Y-axis, and 31 z for a Z-axis), a coil selection circuit 36, an RF receiver 32, an RF transmitter 33, and a sequence controller 34.

The console 40 includes processing circuitry 45, a memory 41, a display 42, and an input interface 43. The console 40 functions as a host computer.

The static magnetic field magnet 10 of the gantry 100 is substantially in the form of a cylinder and generates a static magnetic field inside a bore into which an object such as a patient is transported. The bore is a space inside the cylindrical structure of the gantry 100. The static magnetic field magnet 10 includes a superconducting coil inside, and the superconducting coil is cooled down to an extremely low temperature by liquid helium. The static magnetic field magnet 10 generates a static magnetic field by supplying the superconducting coil with an electric current provided from a static magnetic field power supply (not shown) in an excitation mode. Afterward, the static magnetic field magnet 10 shifts to a permanent current mode, and the static magnetic field power supply is separated. Once it enters the permanent current mode, the static magnetic field magnet 10 continues to generate a strong static magnetic field for a long time, for example, over one year.

The gradient coil 11 is also substantially in the form of a cylinder and is fixed to the inside of the static magnetic field magnet 10. This gradient coil 11 applies gradient magnetic fields (for example, gradient pulses) to the object in the respective directions of the X-axis, the Y-axis, and the Z-axis, by using electric currents supplied from the gradient coil power supplies 31 x, 31 y, and 31 z.

The bed body 52 of the bed 50 can move the table 51 in the vertical direction and in the horizontal direction. The bed body 52 moves the table 51 with an object placed thereon to a predetermined height before imaging. Afterward, when the object is imaged, the bed body 52 moves the table 51 in the horizontal direction so as to move the object to the inside of the bore.

The WB body coil 12 is shaped substantially in the form of a cylinder so as to surround the object and is fixed to the inside of the gradient coil 11. The WB coil 12 applies RF pulses transmitted from the RF transmitter 33 to the object. Further, the WB coil 12 receives magnetic resonance signals, i.e., MR signals emitted from the object due to excitation of hydrogen nuclei.

The MRI apparatus 1 may include the RF coils 20 as shown in FIG. 2 in addition to the WB coil 12. Each of the RF coils 20 is a coil placed close to the body surface of the object. There are various types for the RF coils 20. For example, as the types of the RF coils 20, as shown in FIG. 2, there are a body coil attached to the chest, abdomen, or legs of the object and a spine coil attached to the back side of the object. As another type of the RF coils 20, for example, there is a head coil for imaging the head of the object. Although most of the RF coils 20 are coils dedicated for reception, some of the RF coils 20 such as the head coil are a type that performs both transmission and reception. The RF coils 20 are configured to be attachable to and detachable from the table 51 via a cable.

The RF transmitter 33 generates each RF pulse on the basis of an instruction from the sequence controller 34. The generated RF pulse is transmitted to the WB coil 12 and applied to the object. An MR signal is generated from the object by the application of one or plural RF pulses. Each MR signal is received by the RF coils 20 or the WB coil 12.

The MR signals received by the RF coils 20 are transmitted to the coil selection circuit 36 via cables provided on the table 51 and the bed body 52. The MR signals received by the WB coil 12 are also transmitted to the coil selection circuit 36.

The coil selection circuit 36 selects MR signals outputted from each RF coil 20 or MR signals outputted from the WB coil depending on a control signal outputted from the sequence controller 34 or the console 40.

The selected MR signals are outputted to the RF receiver 32. The RF receiver 32 performs analog to digital (AD) conversion on the MR signals, and outputs the converted signals to the sequence controller 34. The digitized MR signals are referred to as raw data in some cases. The AD conversion may be performed inside each RF coil 20 or inside the coil selection circuit 36.

The sequence controller 34 performs a scan of the object by driving the gradient coil power supplies 31, the RF transmitter 33, and the RF receiver 32 under the control of the console 40. When the sequence controller 34 receives raw data from the RF receiver 32 by performing the scan, the sequence controller 34 transmits the received raw data to the console 40.

The sequence controller 34 includes processing circuitry (not shown). This processing circuitry is configured as, for example, a processor for executing predetermined programs or configured as hardware such as a field programmable gate array (FPGA) or an application specific integrated circuit (ASIC).

The console 40 includes the memory 41, the display 42, the input interface 43, and the processing circuitry 45 as described above.

The memory 41 is a recording medium including a read-only memory (ROM) and a random access memory (RAM) in addition to an external memory device such as a hard disk drive (HDD) and an optical disc device. The memory 41 stores various programs executed by a processor of the processing circuitry 45 as well as various types of data and information.

The input interface 43 includes various devices for an operator to input various types of information and data, and is configured of a mouse, a keyboard, a trackball, and/or a touch panel, for example.

The display 42 is a display device such as a liquid crystal display panel, a plasma display panel, and an organic EL panel.

The processing circuitry 45 is a circuit equipped with a central processing unit (CPU) and/or a special-purpose or general-purpose processor, for example. The processor implements various functions described below by executing the programs stored in the memory 41. The processing circuitry 45 may be configured as hardware such as an FPGA and an ASIC. The various functions described below can also be implemented by such hardware. Additionally, the processing circuitry 45 can implement the various functions by combining hardware processing and software processing based on its processor and programs.

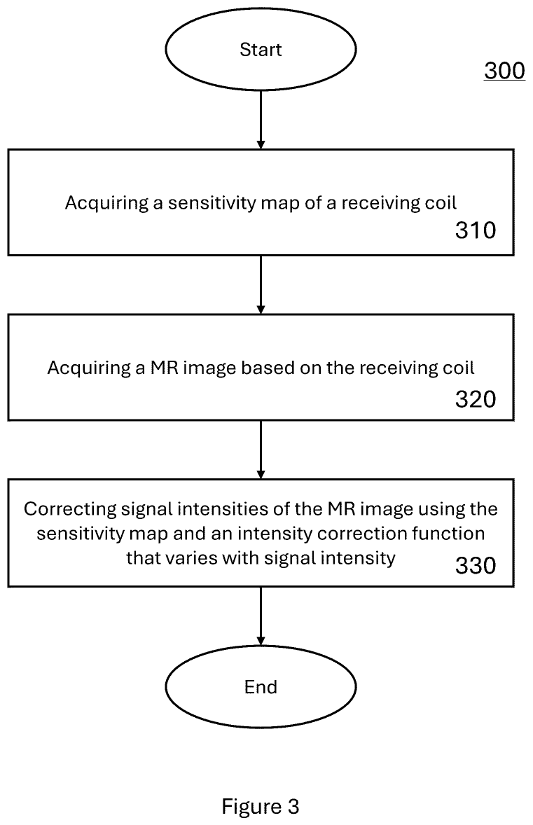

FIG. 3 is a flowchart showing a generalized process as described herein. In method 300, the process begins in step 310 by acquiring a uniformity function (e.g., a sensitivity map) of a receiving coil. In one embodiment, a sensitivity map is used as the uniformity function, and the sensitivity map is generated using a two-part prescan process. In one part, a first image is generated from a whole-body coil (WBC) which is assumed to have a uniform receiver intensity. In the other part of the prescan, a phased array coil (PAC) is used to generate a second image. The sensitivity map (S(x)) is then generated according to Eq. (2) above. Alternatively, the PAC map can be normalized by the sum-of-squares of all channels or by unity (1.0 value everywhere).

In step 320, an MR image is acquired based on the receiving coil. In step 330, the signal intensities of the MR image are corrected using the sensitivity map and an intensity correction function that varies with signal intensity. By using an intensity correction function that varies with signal intensity, noise amplification can be reduced for low signal regions (e.g., corresponding to background (air) or regions of intentionally suppressed tissue signal (FLAIR or FatSat)). This intensity correction function does not change the tissue signal or the tissue SNR. Rather, the intensity correction function improves the visual appearance of the image quality by reducing noise amplification in regions that are expected to be dark or black (i.e., have values of zero). The method varies the correction factor based on the signal intensity of the image. In this way, low signal intensity regions (corresponding to air or suppressed tissue) will be multiplied by a lower factor than they would be normally. Medium or high signals (corresponding to tissue) are multiplied by the regular intensity correction factor and are thus unaffected.

In one embodiment, the intensity correction function (for unsigned data) receives the image itself as an input, such that the correction of an image is given by:

$${I}_{c orrected} ( x ) = \frac{{I}_{o r i g i n a l} ( x )}{S ( x )} * β ( {I}_{o r i g i n a l} ( x ) ) \qquad ( Eq . 4 )$$

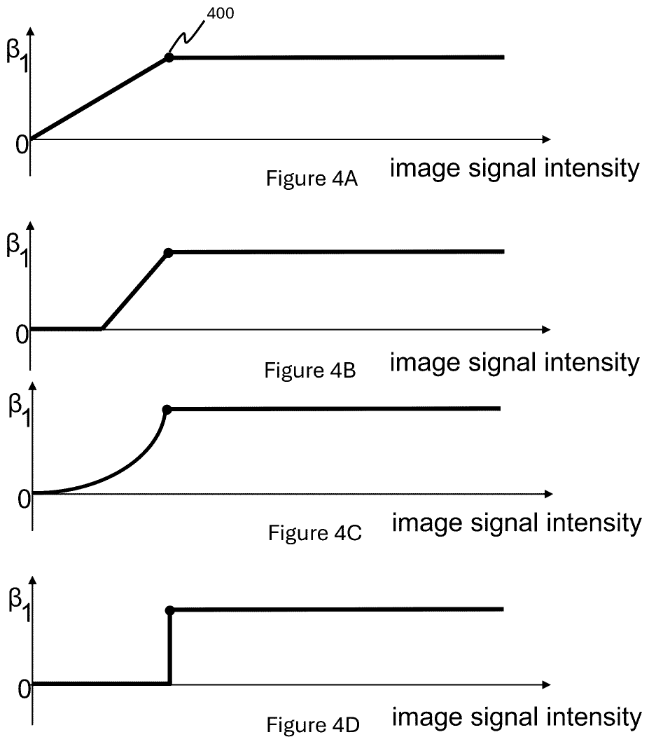

For high signal intensity, β should equal one so that tissue signals are unaffected, otherwise, if β does not equal one within the tissue, the intensity correction function would cause a change in image contrast. FIGS. 4A and 4D show exemplary intensity correction functions. As shown therein, the functions can be (piecewise) continuous (FIG. 4A to FIG. 4C) or discontinuous (FIG. 4D), and the portions before the statically chosen critical transition point 400 can be either linear (ramp) (FIGS. 4A and 4B) or non-linear (e.g., a smoothed curve) (FIG. 4C). Severe intensity correction functions (e.g., Heaviside functions) can introduce images that appear unnatural, so continuous intensity correction functions are preferred.

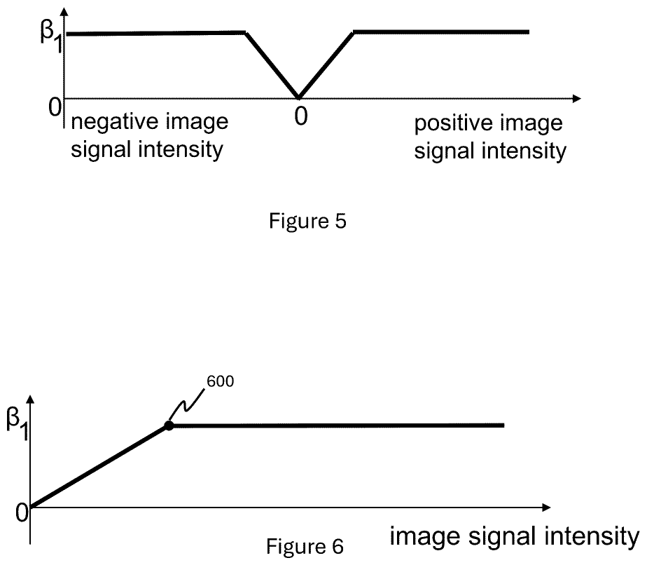

For the case of complex and/or signed magnitude data, the modulation function, as shown in FIG. 5, may look like a ‘notch’ where high positive or high negative values (corresponding to tissue) have a modulation value of β=1 and signal values close to zero (either positive or negative) have a value of β<1.

Alternatively, a data-dependent critical transition point 600 can be dynamically calculated to determine an intensity value at which the intensity correction function should begin having a value of 1.0. In one such embodiment, the critical transition point is determined by an Otsu threshold operation on the uncorrected (original) image. For example, using C++ notation, an intensity value for the critical transition point P may be obtained by:
P=OtsuMethod::GetLevel(magImage, dataSize, 0.15, 512),
where 0.15 represents a maximum threshold value to use, and 512 represents the number of bins used in the calculation. Alternatively, the threshold can be set based on an estimation of the noise level using a prescan measurement of noise.

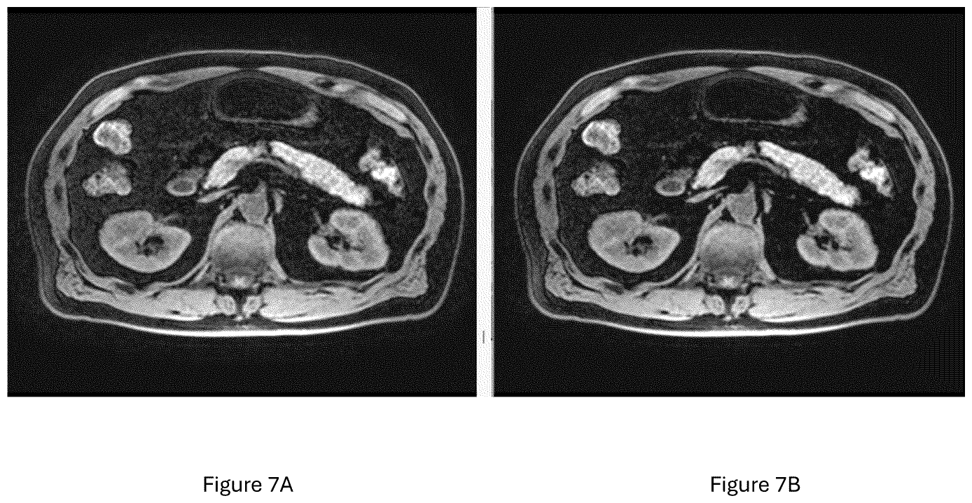

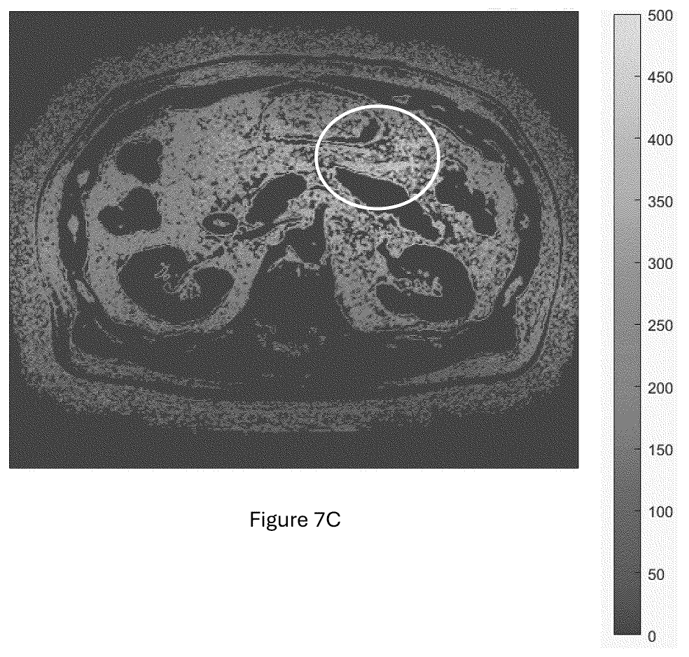

The method reduces the appearance of noise in regions of low signal intensity (e.g., air or suppressed tissue signal), but it does not improve or change the SNR of the issue. However, due to the visual noise suppression, the method improves the perception of image quality and creates more visually pleasing images. For example, FIGS. 7A and 7B are images generated (a) using only a sensitivity map and (b) using a sensitivity map and an intensity correction function, respectively. The image of FIG. 7B has darker areas both inside and outside the body, thereby creating a more pleasing image. FIG. 7C is a color difference image showing differences in signal intensities between FIGS. 7A and 7B. The largest advantage difference is in the circled area corresponding to central tissue where sensitivity is weakest and where the noise amplification would be strongest. The method also improves the ‘halo’ of noise in the air surrounding the body but does not affect tissue signal or tissue SNR (difference=0 in tissue).

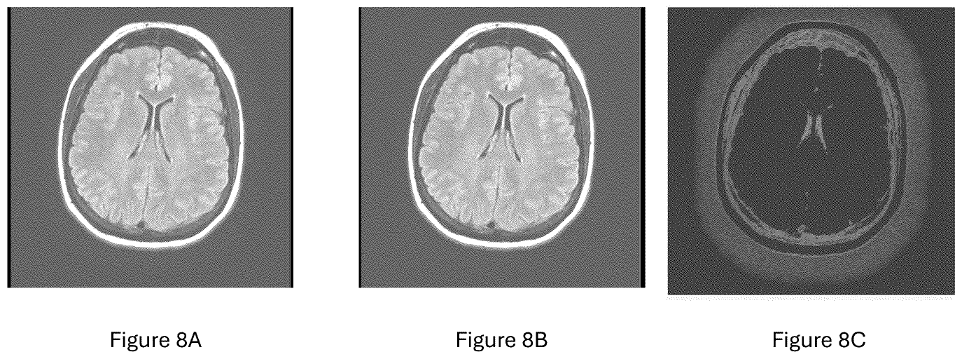

Similarly, FIGS. 8A and 8B are images generated (a) using only a sensitivity map and (b) using a sensitivity map and an intensity correction function, respectively. The image of FIG. 8B has darker areas both inside and outside the body, thereby creating a more pleasing image. FIG. 8C is a color difference image showing differences in signal intensities between FIGS. 8A and 8B.

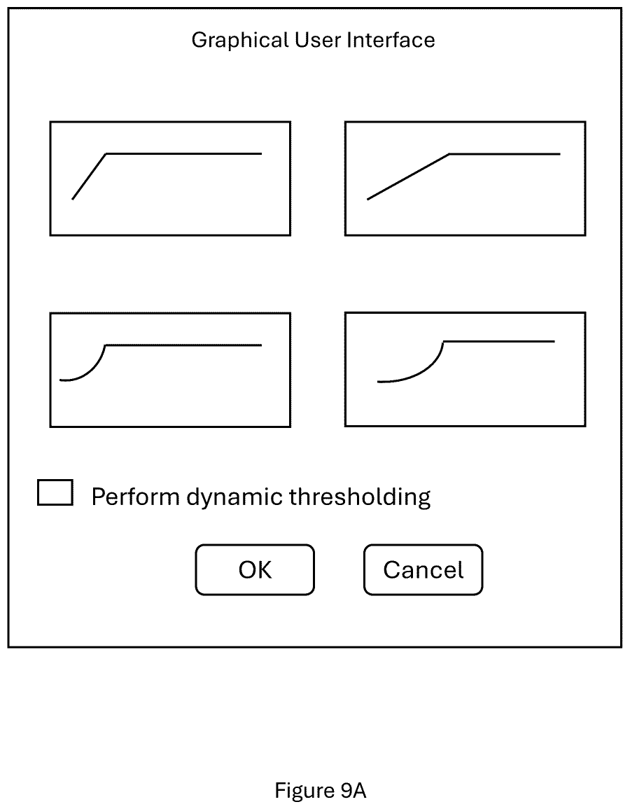

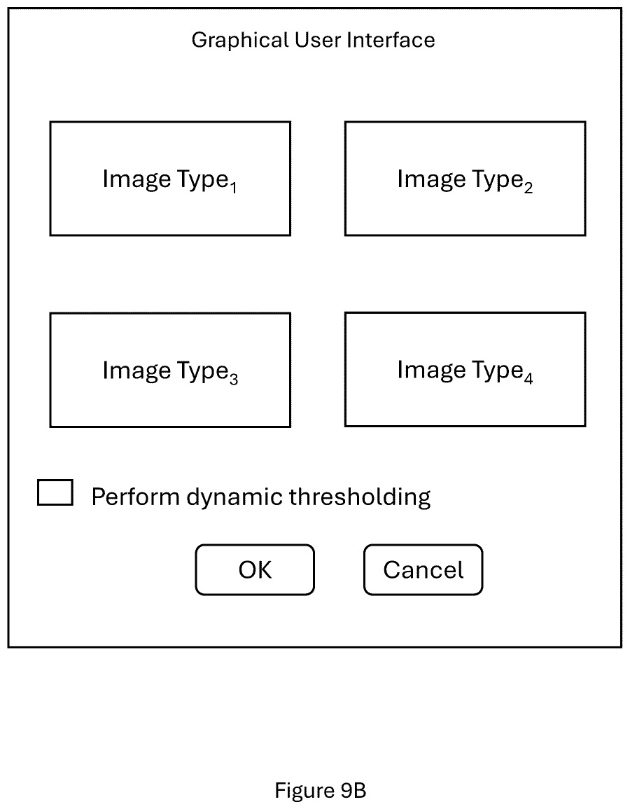

FIGS. 9A and 9B are illustrations of graphical user interfaces for selecting an intensity correction function to be applied to an image that is being corrected. In FIG. 9A, a user may select from a set of graphical illustrations that depict the type of intensity correction function to be applied (e.g., fast linear function, slow linear function, fast non-linear function, and slow non-linear function). The user interface also may include a checkbox or other control for specifying whether the critical transition point is to be determined statically or dynamically (e.g., using an Otsu threshold function).

Alternatively, as shown in FIG. 9B, a user may select from a set of textual descriptions that describe the type of image to which the intensity correction function is to be applied (e.g., brain, hip, or lung). The user interface also may include a checkbox or other control for specifying whether the critical transition point is to be determined statically or dynamically (e.g., using an Otsu threshold function). In yet another embodiment, the image type is automatically detected from at least one of: (1) the image itself (e.g., using a trained neural network that was trained with images and known image types) and (2) data stored with the acquired image data (e.g., data type information stored in DICOM files). Based on the determined image type, the system automatically can apply a corresponding intensity correction function known to be appropriate to that image type.

The methods and systems described herein can be implemented in a number of technologies but generally relate to imaging devices and processing circuitry for performing the processes described herein. In one embodiment, the processing circuitry (e.g., image processing circuitry and controller circuitry) is implemented as one of or as a combination of: an application specific integrated circuit (ASIC), a field programmable gate array (FPGA), a generic array of logic (GAL), a programmable array of logic (PAL), circuitry for allowing one-time programmability of logic gates (e.g., using fuses) or reprogrammable logic gates. Furthermore, the processing circuitry can include a computer processor and having embedded and/or external non-volatile computer readable memory (e.g., RAM, SRAM, FRAM, PROM, EPROM, and/or EEPROM) that stores computer instructions (binary executable instructions and/or interpreted computer instructions) for controlling the computer processor to perform the processes described herein. The computer processor circuitry may implement a single processor or multiprocessors, each supporting a single thread or multiple threads and each having a single core or multiple cores.

Embodiments of the present disclosure may also be as set forth in the following parentheticals.

(1) An image processing method including, but not limited to: acquiring a sensitivity map of a receiving coil; acquiring a MR image based on the receiving coil; and correcting signal intensities of the MR image using the sensitivity map and an intensity correction function, wherein the intensity correction function varies with the signal intensities of the MR image.

(2) The method of (1), wherein the sensitivity map is generated by normalizing measurements obtained from a phased array coil with measurements obtained from a whole-body coil.

(3) The method according to either (1) or (2), wherein the intensity correction function has a maximum value, a minimum value, and is a continuous function of signal intensity.

(4) The method according to (3), wherein the intensity correction function is a linear ramp extending to a critical transition point, after which the value of the function is the maximum value.

(5) The method according to (3), wherein the intensity correction function is a smoothed curve ramp extending to a critical transition point, after which the value of the function is the maximum value.

(6) The method according to (4), wherein the critical transition point of the modulation function is determined by a threshold operation on the signal intensities of the MR image.

(7) The method according to (5), wherein the critical transition point of the modulation function is determined by a threshold operation on the signal intensities of the MR image.

(8) The method according to any one of (3)-(6), wherein the maximum value is 1.

(9) The method according to any one of (3)-(6), wherein the minimum value is 0.

(10) The method according to any of (1)-(3), wherein a shape of the intensity correction function is set by an operator.

(11) The method according to any of (1)-(3), wherein a shape of the intensity correction function is selected based on a type of tissue being imaged.

(12) The method according to any of (1)-(3), wherein a shape of the intensity correction function is selected based on an image acquisition type for the MR image.

(13) An image processing apparatus including, but not limited to: processing circuitry configured to perform the steps of any one of (1)-(12).

(14) A non-transitory computer-readable storage medium storing computer-readable instructions that, when executed by a computer, cause the computer to perform an image processing method of any one of (1)-(12).

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting, of the scope of the disclosure, as well as other claims. The disclosure, including any readily discernible variants of the teachings herein, defines, in part, the scope of the foregoing claim terminology such that no inventive subject matter is dedicated to the public.

1. An image processing method comprising:
acquiring a sensitivity map of a receiving coil;
acquiring a MR image based on the receiving coil; and
correcting signal intensities of the MR image using the sensitivity map and an intensity correction function,
wherein the intensity correction function varies with the signal intensities of the MR image.

2. The method as claimed in claim 1, wherein the sensitivity map is generated by normalizing measurements obtained from a phased array coil with measurements obtained from a whole-body coil.

3. The method as claimed in claim 1, wherein the intensity correction function has a maximum value, a minimum value, and is a continuous function of signal intensity.

4. The method as claimed in claim 3, wherein the intensity correction function is a linear ramp extending to a critical transition point, after which the value of the function is the maximum value.

5. The method as claimed in claim 3, wherein the intensity correction function is a smoothed curve ramp extending to a critical transition point, after which the value of the function is the maximum value.

6. The method as claimed in claim 4, wherein the critical transition point of the modulation function is determined by a threshold operation on the signal intensities of the MR image.

7. The method as claimed in claim 5, wherein the critical transition point of the modulation function is determined by a threshold operation on the signal intensities of the MR image.

8. The method as claimed in claim 1, wherein a shape of the intensity correction function is set by an operator.

9. The method as claimed in claim 1, wherein a shape of the intensity correction function is selected based on a type of tissue being imaged.

10. The method as claimed in claim 1, wherein a shape of the intensity correction function is selected based on an image acquisition type for the MR image.

11. An image processing apparatus comprising:
processing circuitry configured to perform:
acquiring a sensitivity map of a receiving coil;
acquiring a MR image based on the receiving coil; and
correcting signal intensities of the MR image using the sensitivity map and an intensity correction function,
wherein the intensity correction function varies with the signal intensities of the MR image.

12. The image processing apparatus as claimed in claim 11, wherein the sensitivity map is generated by normalizing measurements obtained from a phased array coil with measurements obtained from a whole-body coil.

13. The image processing apparatus as claimed in claim 11, wherein the intensity correction function has a maximum value, a minimum value, and is a continuous function of signal intensity.

14. The image processing apparatus as claimed in claim 13, wherein the intensity correction function is a linear ramp extending to a critical transition point, after which the value of the function is the maximum value.

15. The image processing apparatus as claimed in claim 13, wherein the intensity correction function is a smoothed curve ramp extending to a critical transition point, after which the value of the function is the maximum value.

16. The image processing apparatus as claimed in claim 14, wherein the critical transition point of the modulation function is determined by a threshold operation on the signal intensities of the MR image.

17. The image processing apparatus as claimed in claim 15, wherein the critical transition point of the modulation function is determined by a threshold operation on the signal intensities of the MR image.

18. The image processing apparatus as claimed in claim 11, wherein a shape of the intensity correction function is set by an operator.

19. The image processing apparatus as claimed in claim 11, wherein a shape of the intensity correction function is selected based on a type of tissue being imaged.

20. A non-transitory computer-readable storage medium storing computer-readable instructions that, when executed by a computer, cause the computer to perform an image processing method comprising:
acquiring a sensitivity map of a receiving coil;
acquiring a MR image based on the receiving coil; and
correcting signal intensities of the MR image using the sensitivity map and an intensity correction function,
wherein the intensity correction function varies with the signal intensities of the MR image.
